(19) United States
(12) Patent Application Publication
(10) Pub. No.: US 2018/0220664 A1
(43) Pub. Date: Aug. 9, 2018

(54) SMOKE GENERATOR CONTAINER FOR APPLYING PHYTOSANITARY PRODUCTS AND METHOD OF MANUFACTURING SAME

(71) Applicant: Fomesa Fruitech, S.L., Beniparrell (ES)

(72) Inventors: Ernesto Conesa Roca, Beniparrell (ES); Eduardo García Breijo, Beniparrell (ES); Nicolás Laguarda Miró, Beniparrell (ES)

(21) Appl. No.: 15/427,579

(22) Filed: Feb. 8, 2017

(51) Int. Cl. A23B 7/144; A61L 2/20

(52) U.S. Cl. CPC A23B 7/144; A23V 2002/00; A61L 2/20

(57) ABSTRACT

A smoke generator container for applying phytosanitary products and/or food additives on fruits and vegetables. The container comprises a recipient, a product to be applied inside of the container, and an ignition system. The product to be applied comprises, by weight, between 5% and 30% of an active ingredient, between 25% and 80% of a phyllosilicate matrix, between 5% and 25% of a polysaccharide, and between 5% and 20% of potassium chlorate. The ignition system comprises an electrical resistor from which exit fireproof cables to be connected to a power source. Also, an installation having a plurality of smoke generator containers and a method of manufacturing a smoke generator container.

### FIELD OF THE INVENTION

The present invention refers to a smoke generator container for applying phytosanitary products and food additives for fruit and vegetables, fungicidal products and others having different activity, especially for use to preserve fruits and vegetables after being harvested. The invention also refers to an installation comprising several smoke generator containers for applying phytosanitary products and/or food additives for fruits and vegetables, and a method for manufacturing said smoke generator container.

There are fungicidal products and other types having diverse activity applicable in both the treatment of empty constructions used for storing and handling fruit and vegetables (cold rooms for preserving or degreening, warehouses, trucks, wagons, etc.), as well as in the direct treatment of the products themselves.

The application of said products is usually done with smoke generator containers that enable them to be dispersed as smoke and which are easy to use.

Spanish patent ES 2135340 discloses a fungicidal product with a given composition for applying with a smoke generator, indicated for environmental disinfection of empty, closed areas or areas that contain stored fruits and vegetables, as well as the method for obtaining said fungicidal product.

Spanish patent ES 2142278, an addition to the main patent ES 2135340, specifically develops the subject matter of the latter and extends its purpose. It is not limited to the fungicidal character of the products, but rather it covers other phytosanitary applications of the products, such as preservatives (antioxidants), insecticides exclusively for agricultural use (post-harvest) or antigermatives for applying to stored potatoes.

These smoke generator containers that, among others, contain a combustible product, have a wick for igniting it. The fuel ignites after igniting the wick. The gaseous combustion products create pressure inside the container, from which they exit projected through an orifice in the center of the lid, dragging behind the active material that is dispersed in the smoke and expands with it, reaching all the corners of the premises.

Using wicks to ignite the containers may cause problems such as lack of combustion of the mixtures, or even flames, which in turn could result in high risks due to the flammability of the materials inside the construction where the product is applied. These risks constitute a very important obstacle to the sale of these mixtures, especially in European Union member states.

### SUMMARY OF THE INVENTION

The subject matter of the present invention, therefore, is to provide a smoke generator container for applying phytosanitary products and/or food additives on fruits and vegetables that solves the noted drawbacks of the prior art.

The invention provides a smoke generator container for applying phytosanitary products and/or food additives on fruits and vegetables that comprises a recipient, inside of which is the product to be applied and a system for igniting the container in which the product to be applied is comprised of the following components:
an active ingredient, having a proportion by weight between 5% and 30%, which comprises one or various of these products: Imazalil, o-phenylphenol, thiophanate-methyl, thiabendazole, prochloraz, fosetyl-Al, chlorpropham, pyrethrins, pyrimethanil, fenhexamid, quaternary ammonium, biguanidine, sorbic acid, potassium sorbate, essential oils, ascorbic acid, citric acid and citrates;
a phyllosilicate matrix having a proportion by weight between 25% and 80%, that comprises one or various of these products: antigorite, chrysotile, kaolinite, vermiculite, talc, pyrophyllite, biotite, muscovite, lepidolite, daisy, glauconite and chlorite;
a polysaccharide, having a proportion by weight between 5% and 25%; and
potassium chlorate, having a proportion by weight between 5% and 20%;

and in which the ignition system of the container comprises an electric resistor, from which exit fireproof cables, susceptible for connecting to a power source.

The invention further provides an installation of smoke generator containers for applying phytosanitary products and or food additives on fruits and vegetables according to the various embodiments described herein, that comprises various smoke generator containers for applying phytosanitary products and/or food additives on fruits and vegetables.

The invention further provides a method for manufacturing a smoke generator container for applying phytosanitary products and/or food additives on fruits and vegetables, according to the various embodiments described herein.

The ignition system provides a way to safely apply the mixture, thereby avoiding the possibility of the mixture failing to ignite due to faults that occur when ignition is produced by the wick, besides preventing flames from appearing, as they could provoke an incident by contacting flammable materials that are usually inside the constructions where the disinfection of the fruits and vegetables is done.

The installation of smoke generator containers of the invention also enables a slow ignition and at a distance of more than one smoke generator container, so as to avoid the operators coming into contact with the product, thereby eliminating a risk factor during the application.

The inclusion of polysaccharide as flammable material makes it possible, once the oxidizing has started by heat, for the heat to be uniformly transmitted to the whole mixture. Furthermore, the potassium chlorate acts as an oxidizing agent that supplies oxygen for the start, and the subsequent continuity of the ignition.

Other advantageous embodiments of the invention are disclosed in the following description.

### BRIEF DESCRIPTION OF THE DRAWINGS

The following gives non-limiting descriptions of example embodiments of the subject matter of the present invention, making reference to the accompanying drawings:

FIG. 1 displays a representation of a smoke generator container of the invention for applying phytosanitary products and/or food additives on fruits and vegetables.

FIG. 2 displays a representation of an installation of smoke generator containers of the invention for applying phytosanitary products and/or food additives on fruits and vegetables.

### DETAILED DESCRIPTION OF THE INVENTION

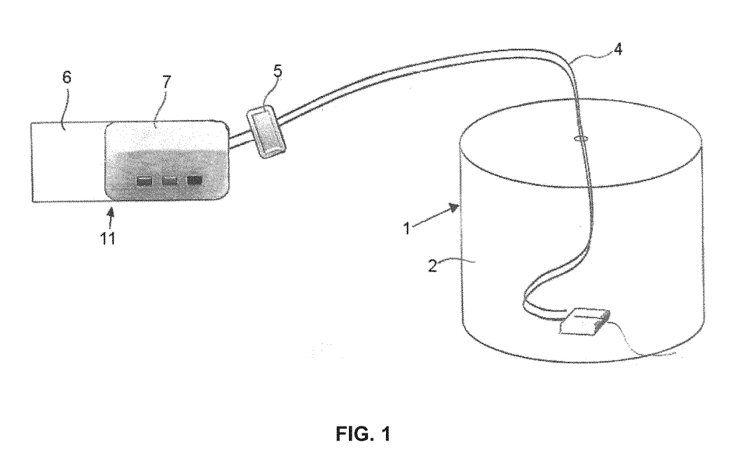

FIG. 1 displays a smoke generator container 1 of the invention for applying phytosanitary products and/or food additives on fruits and vegetables.

The smoke generator container comprises a recipient 2 with a phytosanitary product and food additives in the interior of which an electrical resistor 3 is placed, from which one of the fireproof cables 4 exits toward the exterior. Said fireproof cables 4 may be connected directly or indirectly to a power source 6. The end of the fireproof cables 4 can have a connection.

The product to be applied is comprised of the following components:
an active ingredient, having a proportion by weight between 5% and 30%, which comprises one or various of these products: Imazalil, o-phenylphenol, thiophanate-methyl, thiabendazole, prochloraz, fosetyl-Al, chlorpropham, pyrethrins, pyrimethanil, fenhexamid, quaternary ammonium, biguanidine, sorbic acid, potassium sorbate, essential oils, ascorbic acid, citric acid and citrates;
a phyllosilicate matrix having a proportion by weight between 25% and 80%, that comprises one or various of these products: antigorite, chrysotile, kaolinite, vermiculite, talc, pyrophyllite, biotite, muscovite, lepidolite, daisy, glauconite and chlorite;
a polysaccharide, having a proportion by weight between 5% and 25%; and
potassium chlorate, having a proportion by weight between 5% and 20%.

As indicated, the active ingredient may contain phytosanitary products (Imazalil, o-phenylphenol, thiophanate-methyl, thiabendazole, prochloraz, fosetyl-Al, chlorpropham, pyrethrins, pyrimethanil, fenhexamid, quaternary ammonium, biguanidine, sorbic acid, potassium sorbate, essential oils, ascorbic acid, citric acid and citrates).

In a preferred embodiment, the product comprises the following components:
an active ingredient, having a proportion by weight of 25%
a phyllosilicate matrix having a proportion by weight of 50%
a polysaccharide, having a proportion by weight of 15%, and
potassium chlorate, having a proportion by weight of 10%.

The electrical resistor 3 can be placed on the lower portion of the recipient and, more specifically, at the bottom of the recipient 2.

The ignition system is based on provoking a heating at the bottom portion of the recipient 2 using a resistor 3 and a power source 6, such as a battery (12V, for example). Due to the joule effect, the resistor 3 can cause a temperature increase in the lower portion of the recipient 2, until reaching the critical temperature for starting combustion. The system enables reaching temperatures greater than 200° C. and igniting the containers 1 in about 5 minutes.

Including polysaccharide as the flammable material makes it possible that once combustion has started with the heat provided by the resistor 3, it is uniformly transmitted throughout the mixture. On the other hand, the potassium chlorate actuates as an oxidizer product that supplies oxygen for start up and subsequent ignition continuity. Therefore, there is no need to have an upper air chamber, as there are in other smoke generator containers of the prior art.

A control system 7 can be placed between the power source 6 and the electric resistor 3. For example, a switch can be placed that activates the ignition of the container 1.

Preferably the power source 6 and the control system 7 are located inside the same integrated module 11.

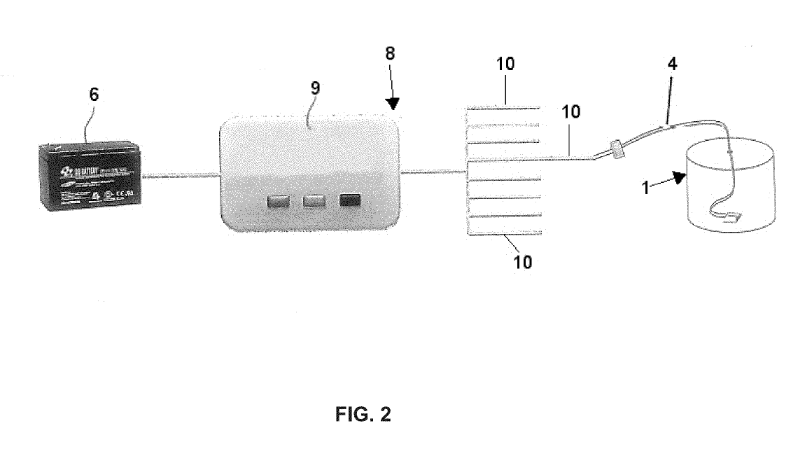

FIG. 2 depicts a representation of an installation 8 of smoke generator containers 1 of the invention for applying phytosanitary products and/or food additives on fruits and vegetables. It is possible to make an electric connection network with several smoke generator containers 1, that enable simultaneous ignition through a single power source 6. In this case the ignition system of the containers 1 comprise a centralized control system 9 with several connections located following the power source 6, so that a fireproof cable 4 exits each connection 10 headed towards each smoke generator container 1.

It is possible to include a programmable automation device like control system 9 that enables igniting diverse smoke generator containers 1 in a determined order. Furthermore, a long distance ignition system would also be possible with a bluetooth type radiofrequency, infrared or mobile phone by using an app.

The manufacturing method of a smoke generator container 1 for applying phytosanitary products and/or food additives on fruits and vegetables of the invention comprises the following steps:
place an electrical resistor 3 in inside the recipient 2,
connect resistor 3 to fireproof cables 4 that, starting from the electrical resistor 3, extend towards the exterior of the recipient 2,
manufacture the product to be applied, which comprises the following steps:
   add the components in the following order: the phyllosilicate matrix, polysaccharide, and the active substance,
   mix, homogenize and mill said mixture,
   add potassium chlorate, and
   mix and homogenize; and
pack the product inside the recipient 2.

Although a few embodiments of the invention have been described and depicted, it is clear that other modifications comprised within the scope thereof can be introduced, and the invention should not be considered limited to said embodiments, but rather only to the content of the following claims.

Claimed is:

1. A smoke generator container for applying at least one of a phytosanitary product, a food additive, or both to a fruit, a vegetable, or both, the smoke generator container comprising:
a recipient,
a product to be applied disposed inside of the container, wherein the product to be applied comprises, by weight:
between 5% and 30% of an active ingredient selected from the group consisting of: Imazalil, o-phenylphenol, thiophanate-methyl, thiabendazole, prochloraz, fosetyl-Al, chlorpropham, pyrethrins, pyrimethanil, fenhexamid, quaternary ammonium, biguanidine, sorbic acid, potassium sorbate, essential oils, ascorbic acid, citric acid and citrates, and combinations thereof;
between 25% and 80% of a phyllosilicate matrix selected from the group consisting of: antigorite, chrysotile, kaolinite, vermiculite, talc, pyrophyllite, biotite, muscovite, lepidolite, daisy, glauconite, chlorite and mixtures thereof;
between 5% and 25% of a polysaccharide; and
between 5% and 20% of potassium chlorate; and,
an ignition system comprising an electrical resistor configured to be connected to a power source with at least one fireproof cable.

2. The smoke generator container according to claim 1, wherein the product to be applied comprises, by weight:
25% of the active ingredient;
50% of the phyllosilicate matrix;
15% of the polysaccharide; and,
10% of potassium chlorate.

3. The smoke generator container according to claim 1, wherein the electrical resistor is located at a bottom portion of the recipient.

4. The smoke generator container according to claim 1 further comprising:
a 12 volt battery as the power source connected to the electrical resistor.

5. The smoke generator container according to claim 4 further comprising:
a control system between the energy source and the electrical resistor.

6. The smoke generator container according to claim 5, wherein the power source and the control system are located in an integrated module.

7. An installation comprising:
a plurality of smoke generator containers for applying at least one of a phytosanitary product, a food additive, or both to a fruit, a vegetable, or both, wherein each smoke generator container comprises:
a recipient,
a product to be applied disposed inside of the container, wherein the product to be applied comprises, by weight:
between 5% and 30% of an active ingredient selected from the group consisting of: Imazalil, o-phenylphenol, thiophanate-methyl, thiabendazole, prochloraz, fosetyl-Al, chlorpropham, pyrethrins, pyrimethanil, fenhexamid, quaternary ammonium, biguanidine, sorbic acid, potassium sorbate, essential oils, ascorbic acid, citric acid and citrates, and combinations thereof;
between 25% and 80% of a phyllosilicate matrix selected from the group consisting of: antigorite, chrysotile, kaolinite, vermiculite, talc, pyrophyllite, biotite, muscovite, lepidolite, daisy, glauconite, chlorite and mixtures thereof;
between 5% and 25% of a polysaccharide; and
between 5% and 20% of potassium chlorate; and,
an ignition system comprising an electrical resistor configured to be connected to a power source with at least one fireproof cable,
wherein the ignition systems of the plurality of smoke generator containers comprise a centralized control system having various connections located following the power source, so that one of the at least one fireproof cables exits each connection towards one of the smoke generator containers from the plurality of smoke generator containers.

8. A method of a manufacturing a smoke generator container for applying at least one of a phytosanitary product, a food additive, or both to a fruit, a vegetable, or both, the smoke generator container comprising:
a recipient,
a product to be applied disposed inside of the container, wherein the product to be applied comprises, by weight:
between 5% and 30% of an active ingredient selected from the group consisting of: Imazalil, o-phenylphenol, thiophanate-methyl, thiabendazole, prochloraz, fosetyl-Al, chlorpropham, pyrethrins, pyrimethanil, fenhexamid, quaternary ammonium, biguanidine, sorbic acid, potassium sorbate, essential oils, ascorbic acid, citric acid and citrates, and combinations thereof;
between 25% and 80% of a phyllosilicate matrix selected from the group consisting of: antigorite, chrysotile, kaolinite, vermiculite, talc, pyrophyllite, biotite, muscovite, lepidolite, daisy, glauconite, chlorite and mixtures thereof;
between 5% and 25% of a polysaccharide; and
between 5% and 20% of potassium chlorate; and,
an ignition system comprising an electrical resistor configured to be connected to a power source with at least one fireproof cable,
wherein the method comprises:
placing the electrical resistor inside the recipient;
connecting the resistor to the at least one fireproof cable that, starting from the electrical resistor, extend towards an exterior of the recipient;
manufacturing the product to be applied, which comprises the following steps:
adding components in the following order: the phyllosilicate matrix, polysaccharide, and the active substance to provide a mixture,
mixing, homogenizing, and milling said mixture,
adding the potassium chlorate, and
mixing and homogenizing; and
packing the product inside the recipient.
